(19) United States
(12) Patent Application Publication
(10) Pub. No.: US 2023/0285505 A1
(43) Pub. Date: Sep. 14, 2023

(54) METHOD OF TREATMENT OF NEUTROPHIL-DRIVEN INFLAMMATORY PATHOLOGIES

(71) Applicant: SUSAVION BIOSCIENCES, INC., Tempe, AZ (US)

(72) Inventors: Laura L. Eggink, Scottsdale, AZ (US); J. Kenneth Hoober, Phoenix, AZ (US)

(21) Appl. No.: 18/007,264

(22) PCT Filed: Jun. 24, 2021
(86) PCT No.: PCT/US21/38999

§ 371 Date: Jan. 27, 2023

Related U.S. Application Data
(60) Provisional application No. 63/057,741, filed on Jul. 28, 2020.

(51) Int. Cl. A61K 38/10; A61K 39/395; A61K 31/573; A61P 17/00

(52) U.S. Cl. CPC A61K 38/10; A61K 39/3955; A61K 31/573; A61P 17/00

(57) ABSTRACT

The present invention provides a method of treating pathological inflammation in a patient comprising: administering to the patient a multivalent structured polypeptide comprising multiple copies of the therapeutic peptide. The present invention also provides a kit, comprising: a multivalent structured polypeptide comprising multiple copies of the therapeutic peptide; and instructions teaching administration of the multivalent structured polypeptide to a patient having atopic dermatitis.

### CROSS REFERENCE TO RELATED APPLICATIONS

This application claims the benefit of U.S. Provisional Patent Application No. 63/057,741, filed on Jul. 28, 2020, the contents of which are incorporated herein by reference in its entirety.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 3,655 byte ASCII (text) file named “Seq_List” created on Jun. 23, 2021.

### TECHNICAL FIELD

This invention relates to the treatment of inflammatory diseases of epithelial tissues caused by excessive neutrophil infiltration, such as atopic dermatitis (eczema), psoriasis, and asthma with therapeutic peptides.

### BACKGROUND

This document describes a unique and effective treatment for neutrophil-driven inflammatory diseases, for example, atopic dermatitis (AD, which encompasses eczema and ichthyosis vulgaris), psoriasis, and asthma.

Atopic dermatitis (AD, eczema) is one of the most common inflammatory disorders affecting up to 20% of children, with age of onset 3 to 5 months, and 10% of adults (Langan et al., 2020; Bitton et al., 2020). The incidence varies between countries, with 4.9% in the US and 2.1% in Japan but 20% in Sweden (Urban et al., 2021). More than 230 million individuals worldwide experience eczema. Approximately 2% (>125 million people) of the world population is affected by psoriasis, an immunogenic disease that in severe cases affects more than 10% of the body (Lowes et al., 2014; Chiang et al., 2019). The strongest genetic risk factors in AD are mutations in the gene encoding filaggrin, although only 20 to 40% of patients have FLG loss-of-function mutations (Smith et al., 2006). Other genetic and environmental factors account for the majority of the cases. In contrast to AD, psoriasis appears later in life, usually early adulthood, and does not improve with age (Guttman-Yassky et al., 2011). The impact on the quality of life of patients and their families by these diseases is profound and multifaceted.

Front-line treatments for AD, particularly for children, include topical corticosteroids and moisturizers to reduce inflammation. A type-2 immune response, with IL-4 and IL-13 as dominant factors, is a primary driver of inflammation. Subcutaneous injection of a monoclonal antibody (dupilumab) has been effective in patients receiving doses every other week (Hamilton et al., 2015; Harb and Chatila, 2020). This antibody binds to IL-4Ra, the common subunit of the receptors for IL-4 and IL-13, which mediates T_{H}2 differentiation and pro-allergic adaptive immune responses. However, significant ocular distress, in particular conjunctivitis, is experienced by about a third of patients who receive the antibody treatment. Several types of immune cells play a role in psoriasis, with IL-23 produced by CD301b⁺ dendritic cells (DCs) playing a pivotal role in stimulating IL-17 production by activated T cells (Lowes et al., 2014; Kim et al., 2017). Munera-Campos and Carrascosa (2019) and Saini and Pansare (2019) listed the many additional antibodies against IL-13, IL-22, IL-33 and other cytokines that are under development and the large number of small molecules being developed for topical or oral applications.

The current activity to achieve effective treatments is an indication of the need for therapies specifically against AD and psoriasis that will decrease the global burden in health care costs and morbidity caused by this malady. Thus, there is a need for a more effective treatment approach to AD.

### SUMMARY

The present invention relates to a method of treating a patient having a neutrophil-driven inflammatory disease, the method comprising: administering to the patient a multivalent structured polypeptide comprising at least two copies of a therapeutic peptide; wherein the sequence of the therapeutic peptide consists of the sequence X₁-X₂-X₃-X₄-X₅-X₆-X₇-X₈-NQHTPR (SEQ ID NO: 10) with each of X₁, X₂, X₃, X₄, X₅, X₆, X₇, and X₈ independently being absent or any amino acid residue, so long as the therapeutic peptide comprises at least 7 amino acid residues and at least one of X₁, X₂, X₃, X₄, X₅, X₆, X₇, and X₈ is Q.

In some aspects, the therapeutic peptide acts as a substrate for a transglutaminase to induce cross-linking of the stratum corneum, restore the epidermal barrier, and protect the patient from environmental pathogens and allergens.

In other aspects, the method further comprises identifying the patient as having a neutrophil-driven inflammatory disease. In one aspect, the neutrophil-driven inflammatory disease is atopic dermatitis (AD), psoriasis, or asthma. In another aspect, the neutrophil-driven inflammatory disease is AD.

In some aspects, at least two of X₁, X₂, X₃, X₄, X₅, X₆, X₇, and X₈ are Q and the at least two amino acid residues of Q are separated by two amino acid residues. In other aspects, the therapeutic peptide consists of 7 to 12 amino acids. In a certain aspect, the therapeutic peptide comprises VQATQSNQHTPR (SEQ ID NO:1).

In yet other aspects, the multivalent structured polypeptide has a central framework, a linker sequence, and at least two arms, wherein each arm comprises the therapeutic peptide, and each arm is linked to the central framework via the linker sequence. In certain aspects, the linker sequence is selected from the group consisting of: GGGS (SEQ ID NO:3), GGGSGGGS (SEQ ID NO:4), SSSS (SEQ ID NO:5), and SSSSSSSS (SEQ ID NO:6). In one aspect, the multivalent structured polypeptide is tetravalent. In another aspect, the multivalent structured polypeptide comprises or consists of svL4 (SEQ ID NO:7).

In some aspects, the multivalent structured polypeptide comprises at least two therapeutic peptides comprising VQATQSNQHTPR (SEQ ID NO:1) and at least one therapeutic peptide comprising NPSHPLSG (SEQ ID NO:2).

In other aspects, the therapeutic peptide is administered topically. In some aspects, the multivalent structured polypeptide is administered to an area where dermatitis is present.

In yet other aspects, the method further comprises administering to the patient at least one topical corticosteroid and/or at least one monoclonal antibody. In one aspect, the at least one topical corticosteroid is selected from the group consisting of triamcinolone acetonide, hydrocortisone, and a combination thereof. In another aspect, the at least one monoclonal antibody is selected from the group consisting of dupilumab, nemolizumab, secukinumab, and combinations thereof.

In certain aspects, the present invention relates to a kit, comprising: a multivalent structured polypeptide comprising at least two copies of a therapeutic peptide; wherein the sequence of the therapeutic peptide consists of the sequence X₁-X₂-X₃-X₄-X₅-X₆-X₇-X₈-NQHTPR (SEQ ID NO: 10) with each of X₁, X₂, X₃, X₄, X₅, X₆, X₇, and X₈ independently being absent or any amino acid residue, so long as the therapeutic peptide comprises at least 7 amino acid residues and at least one of X₁, X₂, X₃, X₄, X₅, X₆, X₇, and X₈ is Q; and instructions teaching administration of the multivalent structured polypeptide to a patient having a neutrophil-driven inflammatory disease.

In some aspects, at least two of X₁, X₂, X₃, X₄, X₅, X₆, X₇, and X₈ are Q and the at least two amino acid residues of Q are separated by two amino acid residues. In other aspects, the therapeutic peptide consists of 7 to 12 amino acids. In a certain aspect, the therapeutic peptide comprises VQATQSNQHTPR (SEQ ID NO:1). In one aspect, the multivalent structured polypeptide comprises or consists of svL4 (SEQ ID NO:7).

In some aspects, the kit further comprises at least one topical corticosteroid and/or at least one monoclonal antibody. In one aspect, the at least one topical corticosteroid is selected from the group consisting of triamcinolone acetonide, hydrocortisone, and a combination thereof; and/or the at least one monoclonal antibody is selected from the group consisting of dupilumab, nemolizumab, secukinumab, and combinations thereof.

In other aspects, the present invention relates to a pharmaceutical composition comprising: a multivalent structured polypeptide comprising at least two copies of a therapeutic peptide; wherein the sequence of the therapeutic peptide consists of the sequence X₁-X₂-X₃-X₄-X₅-X₆-X₇-X₈-NQHTPR (SEQ ID NO: 10) with each of X₁, X₂, X₃, X₄, X₅, X₆, X₇, and X₈ independently being absent or any amino acid residue, so long as the therapeutic peptide comprises at least 7 amino acid residues and at least one of X₁, X₂, X₃, X₄, X₅, X₆, X₇, and X₈ is Q; and a second pharmaceutical intervention, wherein the second pharmaceutical intervention is a corticosteroid and/or a monoclonal antibody.

In one aspect, the multivalent structured polypeptide comprises or consists of svL4 (SEQ ID NO:7).

In some aspects, the second pharmaceutical intervention comprises a topical corticosteroid selected from triamcinolone acetonide, hydrocortisone, and a combination thereof.

In other aspects, the second pharmaceutical intervention comprises an injectable monoclonal antibody selected from the group consisting of dupilumab, nemolizumab, secukinumab, and combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, and 1C depict the skin of naïve C57BL/6 mice. FIG. 1A depicts the hematoxylin and eosin stain showing the thin, violet-colored epidermis with intensely staining nuclei in cells of the basal layer. The intensity of pink stain of the dermis indicates the level of collagen. FIG. 1B depicts application of anti-Ly6G revealing a dermis essentially free of neutrophils. FIG. 1C depicts the dermis, which is richly populated with cells (DCs and macrophages) that stain with an antibody against CD301b (MGL2). The bar represents 100 μm.

FIGS. 2A-2F depict changes in the morphology of skin during treatment with svL4. Images were obtained at the end of a 14-day study. Depilation resulted in (FIG. 2A) a thick epidermis and (FIG. 2B) an abundance of neutrophils in the dermis underlying lesions. A 2-h treatment with 1% SDS every 3 days followed by addition of LPS to the daily PBS dressing resulted in (FIG. 2C) frequent necrotic lesions within a thick epidermis and (FIG. 2D) a very high frequency of neutrophils. Inclusion of 1 μM svL4 in the LPS-containing dressing resulted in (FIG. 2E) normal morphology, with a thin epidermis and a more intense collagen stain in the dermis, which (FIG. 2F) was essentially free of neutrophils. The bar represents 100 μm.

FIGS. 3A and 3B depict graphical representations of the images shown in FIGS. 2A-2F. FIG. 3A depicts epidermal thickness measured at 10 sites along a section of skin for each animal (n=4 in each group). FIG. 3B depicts the counting of neutrophils within 5 separate, defined areas of the dermis of a section of each animal as analyzed in FIG. 3A. Error bars indicate S.E.M.

FIGS. 4A-2H depict sections of skin treated for 5 days with 2 μM svL4 after induction of dermatitis with Staphylococcal enterotoxin B (SEB) and house dust mite extract (HDM). FIGS. 4A, 4C, 4E and 4G depict sections stained with anti-Ly6G to reveal the number of neutrophils.

FIGS. 4B, 4D, 4F and 4H depict sections stained with anti-CD301b. FIGS. 4A and 4B depict sections from the same animal treated with PBS after induction of dermatitis revealing extensive necrosis and a dense population of neutrophils in the epidermis and underlying dermis. FIGS. 4C and 4D depict sections from the same animal treated with svL4 showing a slightly thickened epidermis and numerous CD301b⁺ cells but few neutrophils in the dermis. FIGS. 4E and 4F depict sections from an animal treated with svL4 plus 1 μM dexamethasone. FIGS. 4G and 4H depict sections from an animal that was treated with 1 μM dexamethasone. The bars represent lengths between 100 and 200 μm.

FIG. 5 depicts the effects of SDS and SDS plus svL4 on the amounts of cytokines in extracts of skin from depilated animals. Control extracts were prepared on day 0, at day 2 and at day 5 (squares). The skin was treated with 1% SDS for 2 h on day 0 and day 3. Extracts were prepared 4 h after the first treatment with SDS and on day 2 and day 5 (triangles), or after treatment with 1 μM svL4 in PBS in addition to SDS (circles).

FIG. 6 depicts toxicokinetic curves for svL4 in the rat. The change in serum concentration of svL4 after the peptide was injected intravenously at a dose of 12.5 μmol/kg body weight is shown on a linear axis (top) and a log-based curve (bottom).

FIGS. 7A and 7B depict the changes in serum concentration after the peptide svH1C (squares) was injected intravenously at a dose of 12.5 μmol/kg body weight as compared with a shorter (5-mer) peptide, sv6B (circles). FIG. 7A depicts the curves on a linear scale. FIG. 7B depicts the curves on a log-based axis.

FIGS. 8A and 8B depicts an assay of svL4 as a substrate for transglutaminase (TGase2). The reaction was performed in microtiter wells with porcine liver transglutaminase and polylysine as acceptor. The assay was performed 4 times with slightly different conditions but with the same result. FIG. 8A depicts the extent of reaction as a function of concentration of svL4 in a mixture containing 0.1 M Tris HCl (pH 7.5) buffer and 10 mM CaCl₂. FIG. 8B depicts the extent of reaction as a function of concentration in a mixture containing 50 mM HEPES (pH 7.2) buffer containing 15 mM CaCl₂. svL4 and sv6D were assayed under the same conditions.

FIG. 9A to 9C depict the structures of tetravalent peptides svH1C (SEQ ID NO:9) (FIG. 9A), sv6D (SEQ ID NO:8) (FIG. 9B), and svL4 (SEQ ID NO:7) (FIG. 9C).

FIG. 10 depicts a space-filling model of an arm of the tetravalent peptide svL4 with the two exposed glutamine residues identified with circles.

FIG. 11 depicts the effect of cross-linking a multivalent peptide with multiple proteins and/or cells, catalyzed by transglutaminase, to form a tight epidermal surface barrier that functions to protect an individual from environmental pathogens and allergens.

### DETAILED DESCRIPTION

In certain aspects of the present invention, we describe a topical treatment for atopic dermatitis by application of a multivalent peptide, svL4, that serves as a functional substrate for transglutaminases (TGases). The additional cross-linking ability that svL4 provides allows closure of lesions and repair of the epidermal surface barrier. Dermatitis was induced on depilated mouse skin with lipopolysaccharide (LPS) after a 2-hour treatment with 1% SDS every third day as a penetration enhancer. The irritants caused thickening of the epidermis, numerous necrotic lesions, and an abundance of neutrophils in the dermis underlying the lesions. When svL4 was included in the topical treatment, neutrophils in the dermis were essentially absent and the skin had returned to its normal morphology after 14 days of treatment. A 5-day treatment with svL4 after induction of dermatitis with an extract of house dust mites and Staphylococcal enterotoxin B resulted in partial resolution, with mostly a thin epidermis, only a few small lesions and a low frequency of neutrophils at the base of the dermis or sub-dermally. svL4 is a mimetic of N-acetylgalactosamine and potentially binds to murine C-type lectin receptor MGL2 (CD301b), the ortholog of human CLEC10A, expressed by dendritic cells (DCs) and macrophages in the dermis. However, the only feature of svL4 that suggests a mechanism for resolution are two glutamine residues in the N-terminal half of each arm of the tetravalent peptide (see FIG. 10). These glutamine residues are substrates for TGase. Thus, in some aspects, restoration of the epidermal barrier function by activation of TGase activity provided by the multifunctional substrate, svL4, restores epidermal morphology and reduction of neutrophils in the dermis. These data support svL4 as a small molecule drug suitable for clinical use.

Murine skin is used extensively to model treatments of AD (Jin et al., 2009; Martel et al., 2017). Mice express two C-type macrophage galactose-type lectin homologues (Higashi et al., 2002), the galactose (Gal)-specific CD301a (MGL1) that is most strongly expressed in macrophages and CD301b (MGL2), the mouse ortholog of the human N-acetylgalactosamine (GalNAc)-specific C-type lectin receptor CLEC10A (CD301) that is a marker for CD1c⁺ DCs (Singh et al., 2009; van Kooyk et al., 2015; Heger et al., 2018; Villani et al., 2017; Brown et al., 2019). Kanemaru et al. (2019) discovered that the NC/Nga strain of mouse has a loss-of-function mutation in the Clec10a gene that encodes MGL1 (CD301a), which leads to susceptibility for AD in response to house dust mites, which contain the primary allergen Der f 2 (Johannessen et al., 2005), a protein that shares a homologous sequence with transglutaminase 3 (TGase3) that serves as an epitope for IgE. Dust mite allergens induced production of proinflammatory cytokines such as IL-6 and TNF-α, a characteristic of dermatitis, which was mediated by toll-like receptor 4 (TLR4). This observation was followed by inducing dermatitis by application of lipopolysaccharide (LPS), a well-known ligand for TLR4. LPS, mediated by CD14, initiates a signaling pathway from TLR4 that leads to activation of NF-κB and release of inflammatory cytokines such as TNF-α, IL-1β, IL-6, IL-8 and IL-12p40 and M1 polarization of macrophages (Lu et al., 2008; Liu et al., 2017). These cytokines are major attractants for neutrophils, which infiltrate the skin at high numbers, particularly in regions where necrotic lesions occur in the epidermis.

We initiated studies to determine whether a small molecule ligand of CD301b would achieve resolution of AD. We tested whether peptides svL4 and sv6D are effective in reducing inflammation in murine skin induced with LPS. We found that topical application of 1 μM svL4 was more effective than 0.1 μM svL4 or 1 μM sv6D. Both are basic peptides, with a positive charge of 10 to 12 at the acidic surface of the stratum corneum (Behne et al., 2002; Hanson et al., 2002). A possible distinguishing factor is the hydrophilicity index (Hopp and Woods, 1981), which is 0.4 for sv6D but 0.1 for svL4. The high positive charge and reduced hydrophilicity of svL4 may facilitate translocation into the skin, which has a net negative charge (Nguyen and Soulika, 2019). However, the particularly important feature that emerged from this study is the role of two glutamine residues that reside near the N-terminus of each arm of the peptide.

In some aspects, the present invention relates to a method of treating neutrophil-driven inflammatory disease in a patient. The neutrophil-driven inflammatory disease may be a respiratory condition or a skin condition.

In some implementations, the patient is administered a multivalent structured polypeptide comprising the therapeutic peptide. In some aspects, the multivalent structured polypeptide comprises at least two copies of the therapeutic peptide. In some embodiments, the multivalent structured polypeptide comprises at least two different therapeutic peptides. In certain embodiments, the multivalent structured polypeptide has a central framework, a linker sequence, and at least two arms. Each arm comprises one therapeutic peptide, and each arm is linked to the central framework via the linker sequence. In certain embodiments, each arm of the multivalent structured polypeptide comprises the same therapeutic peptide. In other embodiments, the arms of the multivalent structured polypeptide do not comprise the same therapeutic peptide. In particular embodiments, the multivalent structured polypeptide has four arms and thus is tetravalent. In some aspects, the linker sequence has a sequence comprising GGGS (SEQ ID NO:3), GGGSGGGS (SEQ ID NO:4), SSSS (SEQ ID NO:5), or SSSSSSSS (SEQ ID NO:6).

In certain embodiments, the multivalent structured polypeptide is tetravalent. In one exemplary embodiment, the multivalent structured polypeptide is svL4 (SEQ ID NO:7), which comprises four arms and each comprises the therapeutic peptide consisting of VQATQSNQHTPR (SEQ ID NO:1).

The methods may further comprise administering to the patient a second pharmaceutical intervention for treating neutrophil-driven inflammatory disease, for example, a steroid or a monoclonal antibody. In some aspects, the monoclonal antibody targets an inflammatory cytokine to decrease the activation of inflammatory pathways.

Also described are compositions and kits for treating neutrophil-driven inflammatory disease in a patient. The composition comprises the therapeutic peptide or the multivalent structured polypeptide described herein. In some embodiments, the composition further comprises a second pharmaceutical intervention for treating neutrophil-driven inflammatory disease. For example, the second pharmaceutical intervention is a steroid or a monoclonal antibody. In some aspects, the monoclonal antibody targets an inflammatory cytokine to decrease the activation of inflammatory pathways. The kits comprise instructions teaching the administration of the therapeutic peptide or the multivalent structured polypeptide.

Particularly distressful inflammatory diseases affect the lung and therefore the ability of patients to breathe effectively. Unfortunately, persistent inflammation in respiratory system frequently leads to some adverse diseases such as asthma, COPD, and pulmonary fibrosis. In fact, neutrophil infiltration in the inflamed lung is considered a hallmark of Acute Respiratory Distress Syndrome (ARDS).

Asthma, ARDS, COPD, and viral infections by coronavirus have serious consequences, which has been the motivation for the major effort to identify drugs for treatments. Infiltration of neutrophils into lung tissue has been identified as a major cause of the inflammation in these conditions. A large number of drugs, many approved for clinical use and others under development, have been tested for ARDS. Some of the monoclonal antibodies used for treatment of inflammatory respiratory conditions were approved for other uses such as AD and psoriasis. Accordingly, in certain implementations, the method of treating neutrophil-driven inflammatory disease in a patient further comprises administering to the subject a second pharmaceutical intervention. In some aspects, the second pharmaceutical intervention is a monoclonal antibody targeting the inflammatory pathways or a leukotriene modifier. Interestingly, corticosteroids are pro-inflammatory in these conditions and are not suitable as therapeutic agents. To improve ease in breathing for the patient, the second pharmaceutical intervention may be a beta agonist, leukotriene modifier, cromolyn sodium, or theophylline.

In particular implementations, the patient is administered the therapeutic peptide or the described composition by inhalation. Accordingly, composition comprising the therapeutic peptide administered to the patient may be aerosolized or in the form of a dry powder. Where the composition comprises the therapeutic peptide in a liquid form, the composition is delivered via a nebulizer so that the therapeutic peptide can be administered by inhalation.

Sanofi developed a monoclonal antibody against the receptor for IL-4 and IL-13, dupilumab (Dupixent), that is successful in treating rashes, asthma, and severe atopic eczema. Dupilumab has emerged as the most successful therapy for allergic diseases including eczema. The antibody binds to IL-4Ra, the common subunit of the receptors for IL-4 and IL-13, which mediates T_{H}2 differentiation and pro-allergic adaptive immune responses. Thus, this antibody mitigates the effects of IL-13 on periostin expression and inhibition of synthesis of filaggrin. However, by blocking the activity of IL-4 and IL-13, dupilumab inhibits alternate activation of macrophages to the CD301b⁺ M2a state and blocks expansion of the IL-10⁺ T_{H}2 population, the loss of which is severely detrimental to the host. The dermal environment induces expression of CD301b in phagocytes independent of IL-4/IL-13 signaling. The ability to down-regulate T_{H}2 inflammation in a variety of disorders led to approval by the Food and Drug Administration (FDA) of dupilumab for the treatment of moderate-to-severe atopic dermatitis, which provides relief for approximately two-thirds of patients after 4 months of subcutaneous injections every other week. However, only 30 to 40% of patients experience clear skin after 12 weeks of treatment.

Another pharmaceutical approved by the FDA for the treatment of dermatitis is crisaborole, which also reduces the effect of IL-4. Crisaborole is a small molecule inhibitor of phosphodiesterase-4, which lowers the level of cyclic-AMP and thereby reduces the release of IL-2, IL-4 and IL-31 and consequently proliferation of T cells. Other therapeutic small molecule drugs for treating dermatitis include macrolide-based inhibitors of calcineurin, such as pimecrolimus and tacrolimus. Although the broad systemic immunosuppressant cyclosporin is effective as a treatment for AD, it has not been licensed in the US or Europe for this purpose.

Several peptides have been studied as treatments for AD. Omiganan, a 12-mer anti-microbial peptide, reduced the Staphylococcus aureus population on the skin but no clinical improvement was observed. A 12-mer derivative of a degradation fragment of serum albumin was found to bind CXCR4, a receptor of SDF-1 (stromal cell-derived factor-1, CXCL12) that is overexpressed in atopic dermatitis, and was an effective therapeutic agent with topical application on mouse skin. This peptide has promise in disrupting the CXCR4/CXCL12 signaling that is involved in cancer and inflammatory diseases. A small molecule product of metabolism of tryptophan in some green vegetables, 3,3′-diindolylmethane, inhibited signaling through the transcriptional factor NF-κB and promoted differentiation of regulatory T cells. Several studies reported the reduction of inflammation by inhibitors of JAK1 and JAK2 such as upadacitinib and ruxolitinib. However, JAK1 and Tyk2 activate STAT3 in response to binding of IL-10 to its receptor, which leads to inhibition of NF-κB and associated expression of pro-inflammatory genes. Thus, inhibition of JAK1 and JAK2 seems counter to an anti-inflammatory goal. Activation of JAK1 by IL-10 also inhibits expression of genes responsive to IL-4 and IL-13 by suppressing activation of STATE. Of particular interest was the finding that application of a lysate of non-pathogenic Gram-negative Vitreoscilla filiformis to the skin of mice activated DCs to produce IL-10 and suppressed skin inflammation in T_{H}2-dominated hypersensitivity in atopic dermatitis in NC/Nga mice. IL-10 is a key regulatory cytokine limiting and ultimately terminating excessive T-cell responses to prevent chronic inflammation and tissue damage.

In some aspects, a second therapeutic agent is combined with the peptides disclosed herein (i.e., administered to the subject concurrently or subsequently). These second therapeutic agents include corticosteroids, betamethasone, tacrolimus, pimecrolimus, narrow-band UVB, PDE4 inhibitors, tofacitinib, dupilumab, and nemolizumab. In a very specific embodiment, the second therapeutic agent is betamethasone, pimecrolimus, or dupilumab.

Psoriatic skin is characterized by high expression of IL-17A and IL-17F, which are involved in neutrophil accumulation. Monoclonal antibodies against IL-17A have shown impressive clinical efficacy in about 50% of patients with psoriasis. Clinical studies with an antibody that neutralizes IL-17C, designated MOR106, were abandoned because of futility in treating AD. However, anti-IL-17 antibodies such as secukinumab (Cosentyx, Novartis), ixekizumab (Taltz, Lilly), and brodalumab (Siliq, Ortho Dermatologics) have shown efficacy in roughly half of treated patients. Antibodies against IL-23 have been developed in the past few years to treat psoriasis. Guselkumab (Janssen) and tildrakizumab (Ilumetri, Sun Pharmaceuticals) have been approved by the FDA. Other antibodies, such as ustekinumab (Janssen), risankizumab (Abbvie) and minkizumab (Lilly) are in clinical trials. Most of these antibodies bind subunit p19 of the IL-23 complex. Similar antibodies are being developed, which have the potential of causing significant immune imbalance or impaired response to a danger signal.

For the treatment of skin conditions, the therapeutic peptide or multivalent structured polypeptide is preferably administered topically to an area where the skin condition is believed to be present. In some aspects, the therapeutic peptide or multivalent structured polypeptide is administered by subcutaneous injection. In other aspects, the therapeutic peptide or multivalent structured polypeptide is administered locally by topical application. In certain aspects where the therapeutic peptide or multivalent structured polypeptide is applied topically, the peptide is incorporated into a cream, ointment, or lotion. In other aspects, the therapeutic peptide is dissolved into a vehicle solvent for topical application. In certain aspects, the peptide is applied to gauze or a bandage that is placed over an area where the skin condition is believed to be present.

In some aspects, the methods of treating a skin condition associated with a neutrophil-infiltration (for example, AD, eczema, or psoriasis) further comprise administering to the patient a second pharmaceutical intervention. The second pharmaceutical intervention may be a steroid, quinoline derivatives, macrolides, azathioprine, cyclophosphamide, cyclosporin A, or tricyclic anesthetic compounds, or a drug targeting the inflammatory pathway like a monoclonal antibody, which is used as an existing treatment for the skin conditions. In some aspects, the steroid is a topical corticosteroid, for example, hydrocortisone, triamcinolone, dexamethasone, prednisone and derivatives, triamcinolone acetonide, betamethasone, clobetasol, fluocinonide, fluocinoline. Some second pharmaceutical interventions are topical creams that use a combination of steroids, for example, triamcinolone acetonide and hydrocortisone. Targeted drugs include macrolide-based calcineurin inhibitors (pimecrolimus, Eidel; and tacrolimus, Protopic) that reduce IL-2 production, phosphodiesterase-4 inhibitors (crisaborole, Eucrisa), and monoclonal antibodies (dupilumab, Dupixent, an anti-IL-4 receptor monoclonal antibody (mAb), and tralokinumab, an anti-IL-13 mAb). Dupixent, which binds to the IL-4Ra subunit, inhibits the action of IL-4 and IL-13. The antibodies are delivered by subcutaneous injection and thus also act on other tissues in the body including the lining of the lungs. In one aspect, the therapeutic peptide and the one or more topical corticosteroids are administered concurrently. In other aspects, the therapeutic peptide and the one or more topical corticosteroids are administered sequentially.

The compositions and kits for comprising a skin condition associated with a neutrophil-infiltration comprise the therapeutic peptide or the multivalent structured polypeptide as described herein. In some embodiments, the composition further comprises a second pharmaceutical intervention for treating neutrophil-driven inflammatory disease. For example, the second pharmaceutical intervention is a steroid or a monoclonal antibody. In some aspects, the monoclonal antibody targets an inflammatory cytokine to decrease the activation of inflammatory pathways.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Experimental Materials and Methods

### Peptides

Synthesis and purification of endotoxin-negative svL4 and sv6D were described previously (Eggink et al., 2018).

### Animals

The studies were conducted at Biomodels LLC, an AAALAC accredited facility in Watertown, Mass. Approval for this work was obtained from Biomodels IACUC (protocol number 17-0613-4). Female C57BL/6J, 10-week old mice were obtained from The Jackson Laboratory (Bar Harbor, Me.) and housed at Biomodels. On day −1, mice were anesthetized with isoflurane and dorsal skin was depilated with electric clippers and chemical treatment with Nair. The skin was then washed with 70% ethanol and PBS. On day 0, mice were again anesthetized and treated for 2 h with 0.2 mL of 1% SDS in a 1 cm² piece of gauze secured with a bio-occlusive dressing (Tegaderm). For naïve mice, sterile water was used instead of SDS. Mice were again anesthetized, the skin was blotted dry, and the skin was covered with gauze containing a mixture of 0.1 mL of 10 μg/mL LPS and 0.1 mL PBS or 2 μM svL4. The treatment solution was replaced every day, whereas the SDS treatment was repeated every three days.

A similar experiment was conducted with Staphylococcal enterotoxin B (100 Toxin Technology, Inc., Sarasota Fla.) and house dust mite extract (10 Greer Laboratories, Inc., Lenoir, N.C.), which were applied to the skin for two days following a 2-hour treatment with SDS. The SDS/allergen treatment was for a second 2-day period. The skin was then washed with PBS and the SDS treatments were continued with 2 μM svL4 applied between SDS treatments for another 5 days.

### Histological Analysis

At the end of the treatment period, mice were euthanized by CO₂ inhalation and a 1 cm² portion of the skin was excised. One-half was fixed in formalin for histopathological analysis while the other half was flash frozen. Histological analysis by H&E staining, measurement of epidermal thickness, and immuno-staining was performed by HistoTox Labs, Inc., Boulder, Colo. Neutrophils were stained on fixed sections with monoclonal anti-Ly6G (RB6-8C5, eBioscience), while frozen samples were prepared for sectioning by embedding in OCT compound, and sections were stained with monoclonal anti-CD301b (11A10-B7, eBioscience). Epidermal thickness of each sample was measured at 10 sites without histological artifacts, perpendicular to the long axis of the sections, 9 to 13 mm in length, and averaged to obtain mean thickness. Data are presented as means±standard of the mean for each treatment. Semi-quantitative severity scores were analyzed by non-parametric T-tests (Mann-Whitney U test). Twin-tailed tests were utilized and significance was set at p≤0.05 for all tests.

### Skin Processing and Cytokine Analysis

After freezing, one skin sample/animal was pulverized and then homogenized to make a 100 mg/mL homogenate, centrifuged to remove insoluble material, and aliquoted into 96 well plates for use in downstream assay. HVEM was assessed using the Mouse TNFRSF14 ELISA Kit (HVEM) (AbCam, Cat #: ab213892). Periostin was assessed using the Mouse Periostin/OSF-2 DuoSet ELISA (R&D Systems, Cat #: DY2955). PDGFc was assessed using the Mouse PDGF-C ELISA Kit (MyBioSource, Cat #: MBS165969). IGF1 was assessed using the Mouse/Rat IGF-I/IGF-1 DuoSet ELISA (R&D Systems, Cat #: DY791). IL-24 was assessed using the Mouse IL-24 DuoSet ELISA (R&D Systems, Cat #: DY2786-05). IL-10, IL-13, and IL27 were assessed using a multiplex ProcartaPlex Assay (ThermoFisher Scientific, Cat #: PPX-03).

### Transglutaminase Assay

Two reaction mixtures (50 μL) were tested in polylysine-coated microtiter wells. The first contained 100 mM Tris HCl, pH 7.5, 10 mM CaCl₂, 5 mM DTT, 1 mM EDTA, 150 mM NaCl and 0.05% Tween-20. The second contained 50 mM HEPES buffer, pH. 7.2, containing 15 mM CaCl₂, 5 mM DTT, 0.5 mM EDTA, 125 mM NaCl and 0.05% Tween-20. Peptides were added to provide a series of concentrations from 0 to 200 μM. TGase2 (10 μUnits) from pig liver (Sigma-Aldrich, St. Louis, Mo.) was added to each well, and after 30 min of incubation, the wells were washed 3-times with water. Then 50 μL of 0.1 μg/mL streptavidin conjugated with horseradish peroxidase was added and incubated 20 min. The wells were then washed 4-times with PBS containing 0.05% Tween-20 and 100 μL of 3,3′,5,5′-tetramethylbenzidine substrate was added. The reaction was allowed to proceed 5 min and then stopped with 50 μL 1 N H2504 and read immediately at 450 nm.

### Induction of Dermatitis

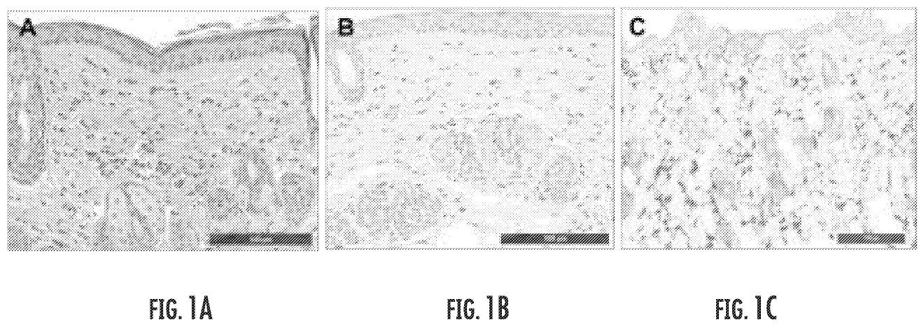

A modification of the model described by Kanemaru et al. (2019) was designed to test activity of the peptides as a treatment for AD. In naïve, healthy murine skin, the epidermis is thin (FIG. 1A) and very few Ly6G⁺ neutrophils are present (FIG. 1B). The dermis contains abundant cells that stain for CD301b⁺, a marker for DCs and macrophages (FIG. 1C) and a potential target for svL4 and sv6D.

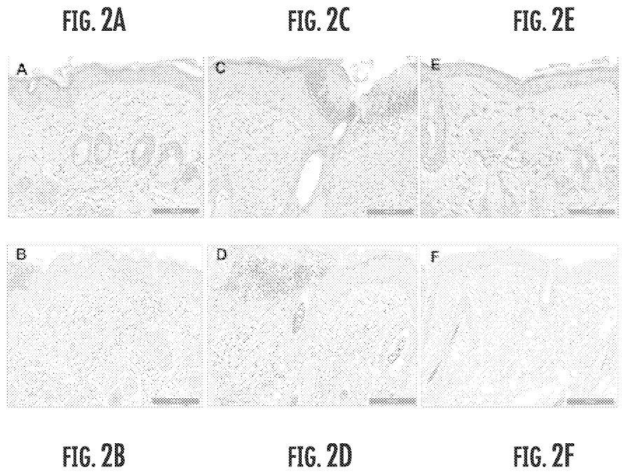

We found that a 2-hour treatment with 4% SDS every three days, as described by Kanemura et al. (2019), caused extensive damage to the skin. Therefore, 1% SDS, which disrupted the barrier function of human skin (Törmä et al., 2008), was used as a penetration enhancer (Karande et al., 2004). On day −1, dorsal skin from mice was shaved, depilated with thioglycolate and sodium hydroxide (Nair), washed with 70% ethanol followed by a wash with PBS. This treatment caused thickening of the epidermis and extensive infiltration of neutrophils without additional irritants when examined at 14 days (FIGS. 2A and 2B). The skin was further treated for 2 h with 1% SDS. The treatment with SDS was repeated every 3 days, with 1-cm² areas of the skin covered with gauze wetted with treatment solution in PBS in the intervening periods. The addition of LPS to the treatment exacerbated the pathology, with a high density of neutrophils in the dermis and in the epidermis within areas of lesions (FIGS. 2C and 2D).

### Example 2. Treatment with svL4 or svH1C Alleviates Atopic Dermatitis

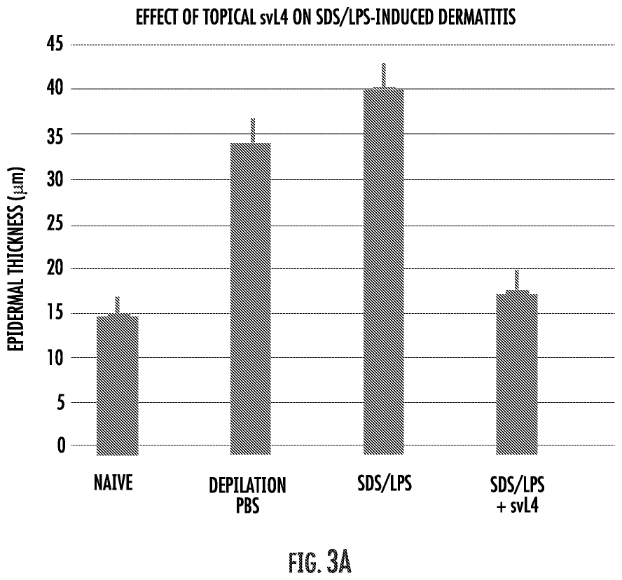

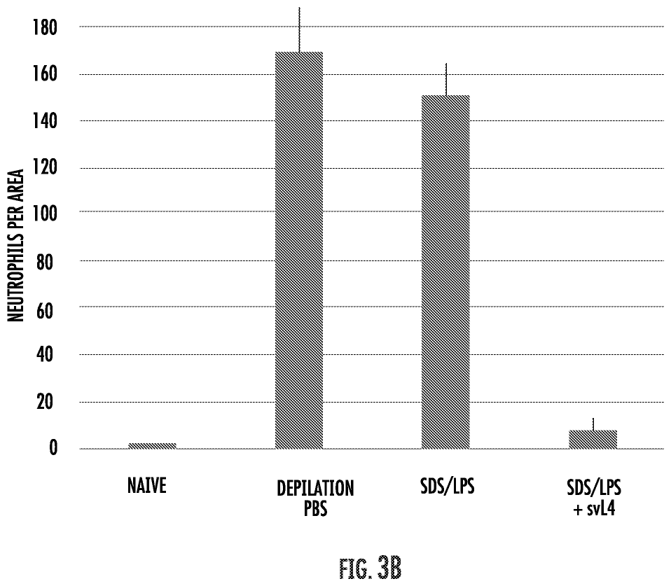

With the possibility that CD301b⁺ cells in the dermis may play a role in resolution of dermatitis, svL4 and sv6D, which are mimetics of N-acetylgalactosamine (GalNAc) (Eggink et al., 2018), were tested for their ability to suppress the morphological features of dermatitis. Resolution was defined as restoration of a thin epidermis and a neutrophil-free dermis. The peptides were tested topically at 0.1, 1.0 or 2.0 μM or with daily subcutaneous injections of 1 nmole/g body weight. svH1C, a mimetic of sialic acid (Eggink et al., 2015), was tested as an alternate peptide. When 1 μM svL4 was applied to the skin in combination with LPS, the epidermis at the end of the 14-day treatment was uniformly of normal thickness and the dermis was nearly free of neutrophils (FIGS. 2E and 2F). svL4-treated skin also lacked necrotic lesions. Graphical representations of epidermal thickness and neutrophil frequency are shown in FIGS. 3A and 3B.

The pathology report indicated that topical application of 1 μM svL4 was the only treatment to overcome the response to the irritants (Table 1). Topical 0.1 μM svL4 was less effective than 1 μM svL4, with thickened epidermis and frequent necrotic lesions (not shown). Interestingly, topical application or subcutaneous injections of 1 μM sv6D, the C-terminal half of svL4 (Eggink et al., 2018), were ineffective. Extensive infiltration of neutrophils was observed with each treatment except for svL4. Although some reduction in the level of dermatitis was observed after subcutaneous injection with 1 μM svH1C, with significant reduction in the abundance of dermal neutrophils, topical application was not effective.

For Table 1, an area of dorsal skin was depilated to provide a 1-cm² surface for treatments with SDS (1%), LPS (1 μg/0.2 mL) and peptide. SDS was applied every third day and dressings containing LPS with or without peptide were replaced daily. The mean epidermal thickness for each treatment was calculated as the average of the means of 10 measurements for each animal in that group. Peptides were applied topically at a concentration of 1 μM and by daily subcutaneous injections at doses of 1 nmole/g. The value for the thickness of the dorsal skin of naïve mice was from Wei et al. (2017). Histopathological analyses identified the frequency of epidermal necrotic lesions, and dermal collagen was characterized by increased density of dermal collagen bundles and more intense eosin staining. Values are ±S.E.M.

TABLE 1
Histopathological parameters of skin after each treatment

| | Epidermis | | Increased |
|---|---|---|---|
| Treatment | Thickness (μm) | Necrosis | Collagen |
| Naïve | 14.9 ± 1.5 | — | — |
| Depilation | 33.6 ± 3.9 | 0.33 ± 0.33 | 0 |
| SDS/LPS | 34.7 ± 4.6 | 0.50 ± 0.22 | 0.83 ± 0.40 |
| SDS/LPS + svL4 | 17.5 ± 0.93 | 0 | 1.50 ± 0.65 |
| (topical) | | | |
| SDS/LPS + svL4 | 33.2 ± 4.7 | 1.25 ± 0.75 | 2.00 ± 0.71 |
| (subcutaneous) | | | |
| SDS/LPS + sv6D | 40.7 ± 6.4 | 0 | 2.00 ± 0.58 |
| (subcutaneous) | | | |
| SDS/LPS + svHIC | 35.4 ± 9.2 | 0.75 ± 0.48 | 1.50 ± 0.29 |
| (topical) | | | |
| SDS/LPS + svH1C | 31.9 ± 7.1 | 0 | 1.50 ± 0.29 |
| (subcutaneous) | | | |

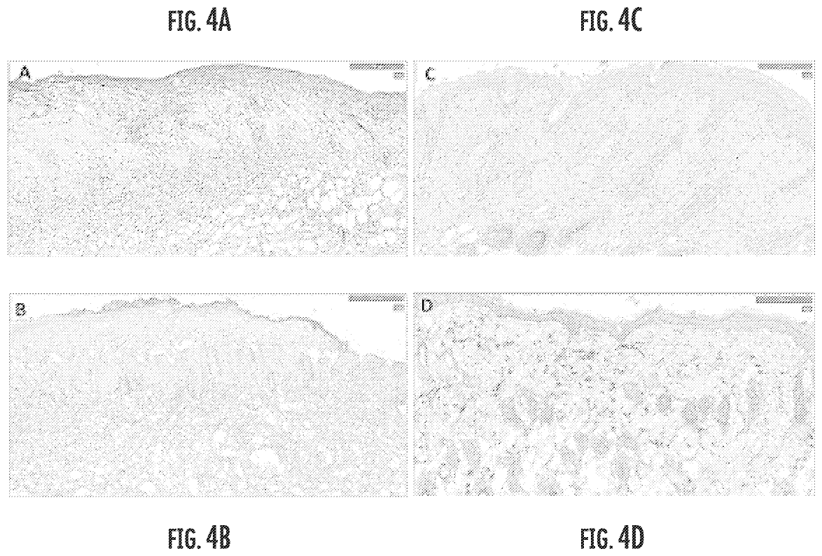

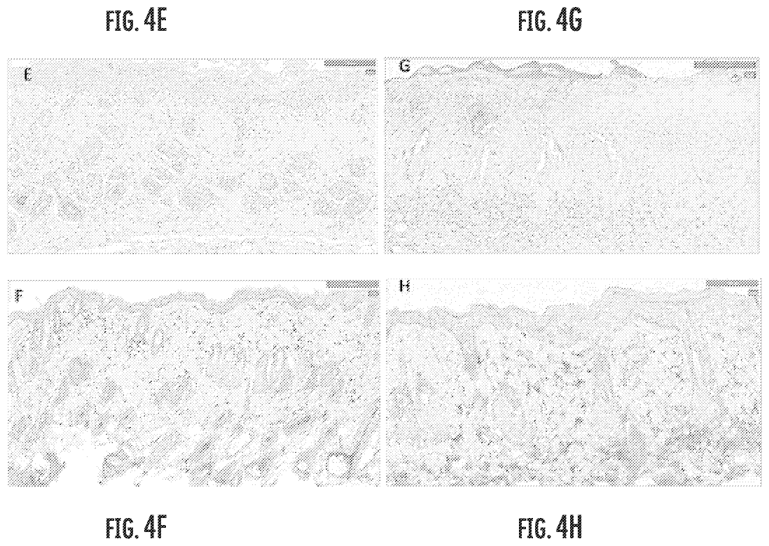

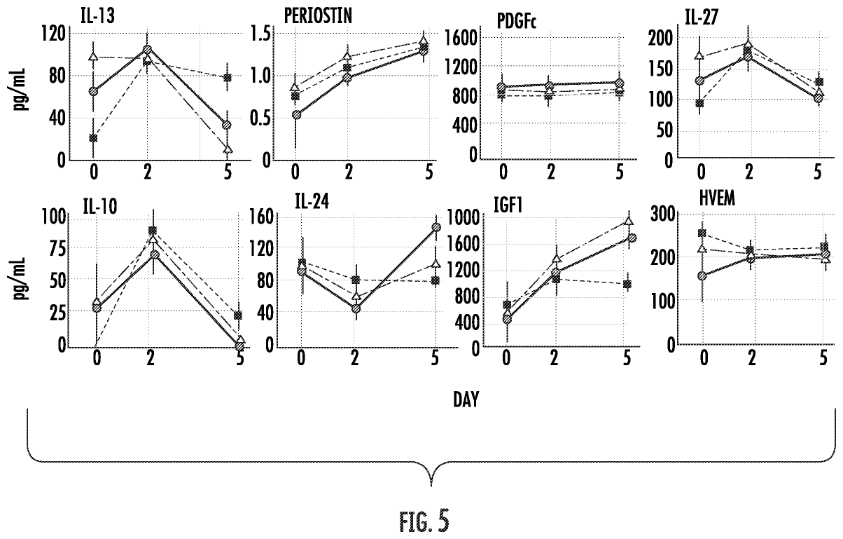

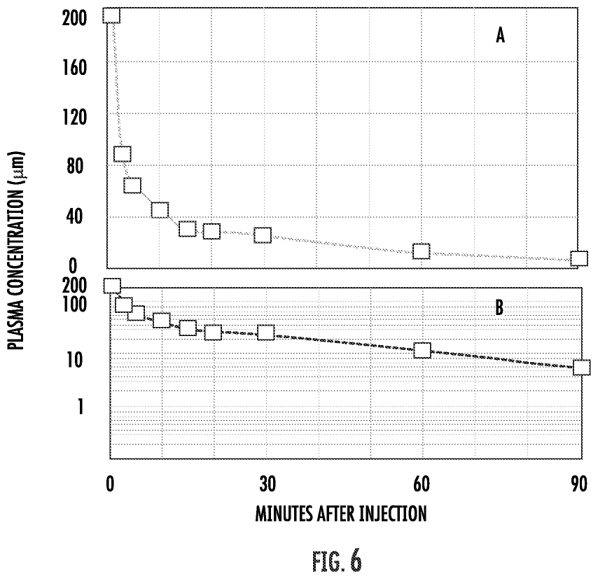

To gain insight into the course of treatment, dermatitis was induced without svL4 over a period of 4 days with 200 μL of a combination of house dust mite extract (HDM, 10 μg) and Staphylococcal enterotoxin B (SEB, 100 μg), which are commonly encountered allergens (Kawakami et al., 2007). Each 2-day treatment with these allergens was preceded by a 2-hour treatment with 1% SDS, as in previous experiments. After the 4-day treatment with the allergens, the skin was treated with 2 μM svL4±1 μM dexamethasone for another 5 days, which was expected to provide an intermediate stage in resolution. Similar to the results shown in FIGS. 3A and 3B, in tissue from animals treated with HDM and SEB, thickened epidermis and extensive necrotic lesions, with an underlying dense population of neutrophils, were evident (FIG. 4A). Treatment with svL4 reduced epidermal thickness to nearly normal, with areas of thickened as well as thin epidermis but with only a few, small lesions (FIGS. 4C and 4D). Neutrophils were nearly absent in the dermis, particularly underlying the lesions, or at a low frequency at the base of the dermis or subdermal region (FIG. 4C). Similar results were obtained with samples from animals treated with svL4 plus dexamethasone (FIGS. 4E and 4F). Treatment with dexamethasone alone showed little improvement over PBS, with several necrotic lesions flanked by areas nearly free of neutrophils (FIGS. 4G and 41I).

CD301b⁺ cells were not detected in the dermis underlying lesions, where the density of neutrophils was high (FIG. 4B). At the end of treatment, svL4-treated skin lacked neutrophils and the abundance of CD301b⁺ cells had recovered to the initial frequency in the dermis below a thin epidermis (FIG. 4D), These events apparently occurred during the resolution phase. CD301b⁺ cells were not detected near lesions during the highly inflammatory phase. The inclusion of dexamethasone in the treatment did not significantly increase the frequency of CD301b⁺ cells (FIG. 4F). Although treatment with dexamethasone alone did not restore the epidermis to normal thickness, CD301b⁺ cells at the base of the dermis appeared more intensely stained (FIG. 41I), which suggested that the receptor was expressed at a higher level (van Vliet et al., 2006).

### Example 3. Cytokine Response to Treatment with svL4

To determine whether other factors may play a role in the resolution of epidermal inflammation, we performed a survey of several key cytokines that have been identified as important in atopic dermatitis and wound healing. For this study, svL4 was applied topically to depilated skin and treated with SDS but without LPS. Cytokines in extracts of skin were measured 4 hours after the first 2-h treatment with SDS, after 2 days and after 5 days, with the treatment with SDS repeated on day 3.

IL-13 plays a dominant role in the lesional skin of atopic dermatitis (Fume et al., 2019; Bitton et al., 2020) and up-regulates periostin and IL-24 (Mitamura et al., 2020). In control samples, the level of IL-13 had increased 2 days after depilation of the skin and remained high at day 5. The level of IL-13 was transiently increased by SDS at day 0 and day 2 but was suppressed by SDS and svL4/SDS at day 5 below that of the depilated skin control. Periostin mediates the IL-13 induction of IL-24, a member of the IL-20 family, which is a subgroup of the IL-10 family of cytokines (Mitamura et al., 2020). Whereas IL-13 levels were lower at day 5, periostin continued to increase with svL4/SDS treatment in parallel with the effect of SDS, which possibly was the cause of the increase in IL-24. IL-24 levels are upregulated in wounds and mediates the effects pro-inflammatory and proliferative effects of IL-13 via periostin but also has an immunosuppressive role in viral infections (Mitamura et al., 2020). The IL-20 family, including IL-24, suppresses production of IL-1β and IL-17A (Mitamura et al., 2020; Myles et al., 2013) and may be a key factor in suppressing keratinocyte proliferation and wound healing (Kolumam et al., 2017; Menezes et al., 2018) and thereby reducing epidermal thickness. After a peak at day 2, release of IL-10 was suppressed (FIG. 5).

Proliferation of the adipocyte precursor subpopulation of myofibroblasts, which is an essential process in wound healing, is induced by platelet-derived growth factor C (PDGFc) and insulin-like growth factor 1 (IGF1) that are produced by CD301b+ macrophages (Shook et al., 2018). We found no change in the level of PDGFc in the skin, regardless of treatment, but IGF1 was increased by svL4 and SDS (FIG. 5). IL-27 stimulates proliferation of keratinocytes, which may be involved in the early thickening of the epidermis (Yang et al., 2017), but was not significantly changed during the initial 5 days of svL4 treatment. These results indicate that svL4 does not have a significant effect on cytokine responses over that of SDS during the early days of treatment, with significant divergence seen only with IL-24.

### Example 4. Acute Toxicity Study

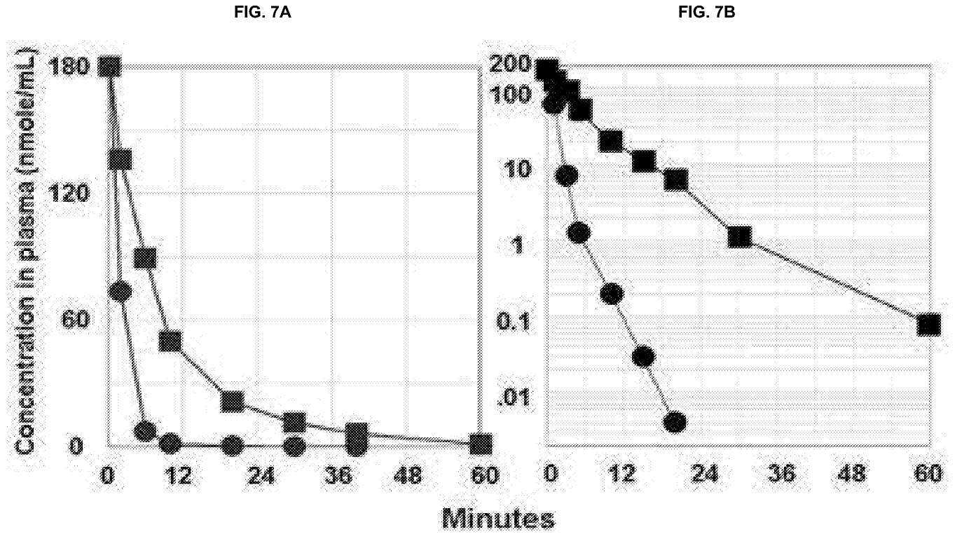

Ten male Hsd: Sprague Dawley®™ SD®™ rats were given peptide svL4 or peptide svHIC at a dose level of 12.5 μmol of test article per kg of body weight at day 1 and day 8 via intravenous injection. This dose was 100-fold higher than a maximal therapeutic dose. The dose volume for each group was 2.5 mL/kg. Assessment of toxicity was based on mortality, clinical signs, body weights, food consumption, clinical pathology, and macroscopic observations. Blood samples were also collected for toxicokinetic evaluation. The change in concentration of svL4 in the serum after the second injection is depicted in FIG. 6. The same analysis was performed for svH1C (FIGS. 7A and 7B) and compared with a shorter (5-mer) peptide, sv6B. Comparison of lifetimes of svL4 and svH1C in serum indicated that the concentration of svL4 in serum 1 hour after injection was about 100-fold greater than that of svH1C, which has a significantly longer lifetime than the shorter sv6B.

No test article-related clinical signs, body weight or body weight change differences, or food consumption alterations were observed. On Day 9, no test article-related effects were present in hematology, coagulation, or clinical chemistry test results of males given 12.5 μmol/kg/dose. No macroscopic lesions were evident at the scheduled necropsy. The peptides had no effect on terminal body weight and none of the organ weight variations were clearly attributable to a test article.

This preliminary toxicity study was performed under contract with Covance Laboratories, Inc., and was designed to demonstrate a margin of safety. Because of the lower bioavailability of peptide administered subcutaneously as compared to intravenous injection, this study should have provided a margin of safety of at least a 1000-fold over a proposed standard therapeutic dose. Administration of peptide svL4 or svH1C suspended in the vehicle control article (standard PBS prepared in sterile, pyrogen-free water) to male rats at a dose of 12.5 μmol/kg/dose using a volume of 2.5 mL/kg was well tolerated. No test article-related findings were noted.

### Example 5. Analysis of Stability/Forced Degradation of Peptides

The purity of the peptides was reported by the manufacturer as >95% and was confirmed independently. The peptides are stable indefinitely in dry form. After purification, peptides were prepared as solutions in PBS or 150 mM NaCl. No significant change occurred in the mass spectrum of peptides when dissolved in PBS and stored for three years at −20° C. with occasional thawing. The mass spectrum obtained after storage of svH1C at 4° C. for 1 year showed no significant deterioration as compared to a spectrum obtained shortly after purification. The molecular mass of svH1C is 4,594 Da. Similar stability was found with svL4 (molecular mass, 6,826 Da).

Stability of the synthetic products was assessed by mass spectroscopy after they were subjected to a specific set of rigorous conditions. A summary of a forced degradation study performed by Blue Stream Laboratories is shown in Table 2.

A study on stability of svL4 in plasma indicated that 55% loss occurred in rat plasma over 48 h at room temperature, whereas only 15% loss occurred in dog plasma. Stability of svH1C in rat and dog plasma indicated that complete degradation occurred in rat plasma over 48 h at room temperature, while 92% loss occurred in dog plasma. For bioanalytical samples, loss of peptide in plasma was prevented by addition of potassium oxalate, NaF and formic acid (2%).

TABLE 2
Stability of peptides, initially dissolved
in PBS, pH 7.4, under stress conditions.

| | |
|---|---|
| | Percent |
| Stress Condition | Remaining |

SvL4

| | |
|---|---|
| Thermal stress: 60° C./ambient relative humidity, | ~60 |
| 14 days | |
| High pH: 40° C., 0.1N NaOH/pH 11, 5 days | 0 |
| Low pH: 40° C., 0.1N HCl/pH 3, 5 days | 100 |
| Oxidation: 40° C., 0.5% H₂O₂, 2 days | 100 |

svH1C

| | |
|---|---|
| Thermal stress: 60° C./ambient relative humidity, | 0 |
| 14 days | |
| High pH: 40° C., 0.1N NaOH/pH 11, 5 days | 0 |
| Low pH: 40° C., 0.1N HCl/pH 3, 5 days | 100 |
| Oxidation: 40° C., 0.5% H₂O₂, 2 days | 50 |

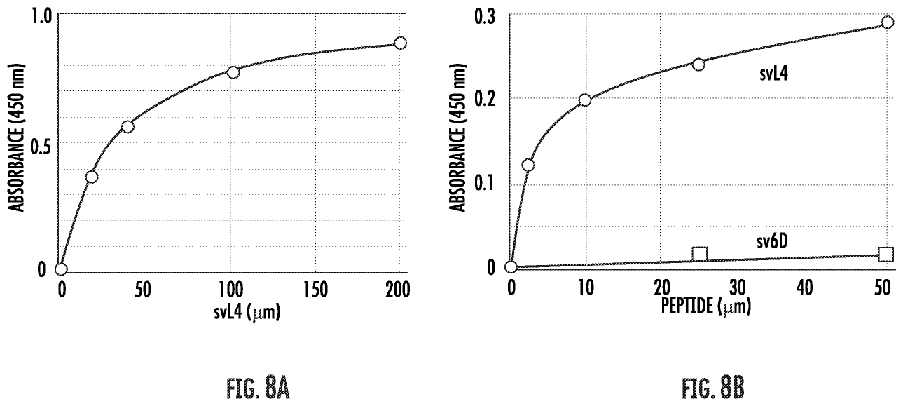

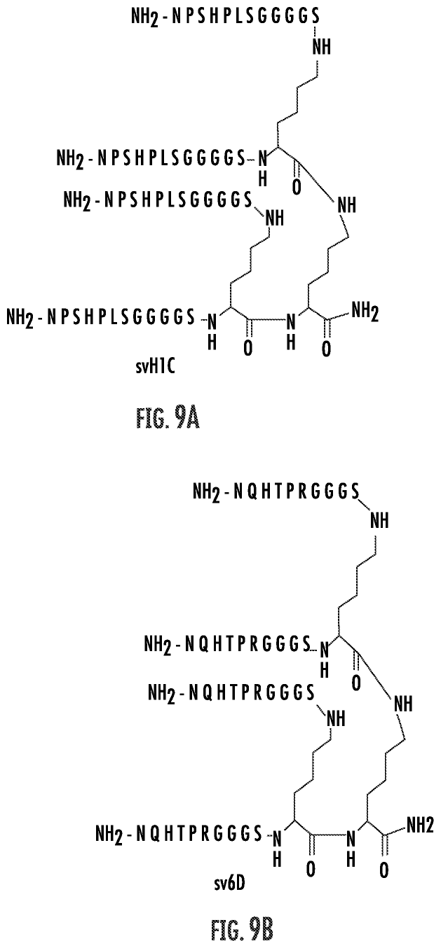

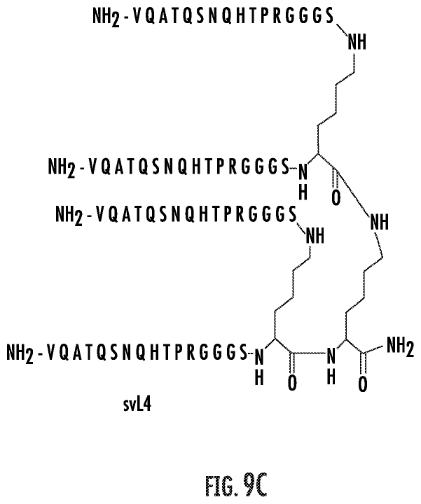

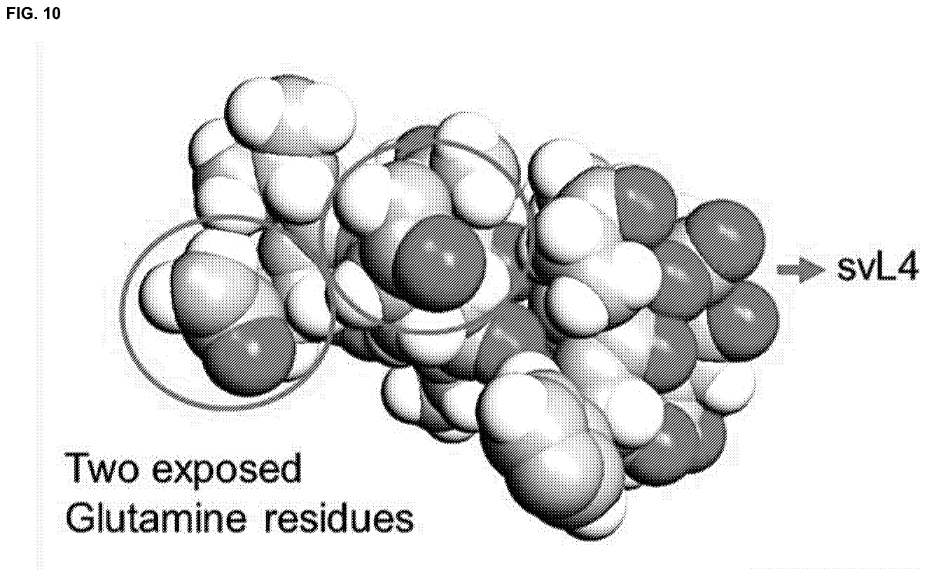

### Example 6. Involvement of Transglutaminase (TGase) in svL4 Resolution of Dermatitis

Whereas svL4 and sv6D are ligands for CD301b expressed by DCs and macrophages, and CD301b⁺ cells were abundant during the resolution phase of therapy, the role—if any, of these cells in the initial restoration phase was not obvious. We thus considered other possibilities. Liedén et al. (2012) and Su et al. (2020) demonstrated a remarkable increase in expression of TGases under the stratum corneum of patients with atopic dermatitis. Because TGase activity is required for formation of a normal epidermal barrier, this upregulation seems to be a response to inflammation and barrier dysfunction. It occurred to us that the glutamine residues in each of the arms of the tetravalent svL4 may provide a substrate for TGases and thereby offer additional cross-linking opportunities. As shown in FIGS. 8A and 8B, the assay with svL4 demonstrated a strong reaction with TGase2. The minimal activity with sv6D as the substrate revealed that the ability of svL4 to resolve dermatitis is solely related to its ability to serve as a substrate for TGase. In this context, the glutamines in svL4 are accessible to the enzyme as substrates (see FIGS. 9C and 10). TGase1 is expressed in the stratum granulosum of the epidermis and is the major enzyme involved in formation of the cornified envelope beneath the plasma membrane of terminally differentiating keratinocytes (Kalinin et al., 2001). TGase1 initially catalyzes attachment of the scaffold protein involucrin to the inner surface of the membrane followed by cross-linking of the major protein of the cornified envelope, loricrin. Reduction of the cell to a collapsed, insoluble physical barrier is accompanied by replacement of the plasma membrane with ceramide lipids that seal the space between cells. Whereas TGase1 is confined to the cellular interior, resolution of normal epidermal morphology by topical application of svL4 implies an extracellular reaction that cross-links cells. It is possible that within necrotic lesions the cellular structures are disrupted sufficiently for the peptide to gain access to TGase1. Alternatively, TGase2, which is expressed ubiquitously, is secreted from cells and is involved in cell adhesion and wound healing (Griffin et al., 2002; Eckert et al., 2005). Thus, TGase2 may provide the critical activity in restoring the surface barrier.

svL4 Provides a TGase Substrate to Enhance Cross-Linking of the Stratum Corneum Thereby Restoring the Epidermal Barrier and Alleviating Atopic Dermatitis

These data suggest that the mechanism of action of svL4 in the restoration of AD is provision of a substrate for TGases to enhance cross-linking of the stratum corneum and to restore the epidermal barrier function.

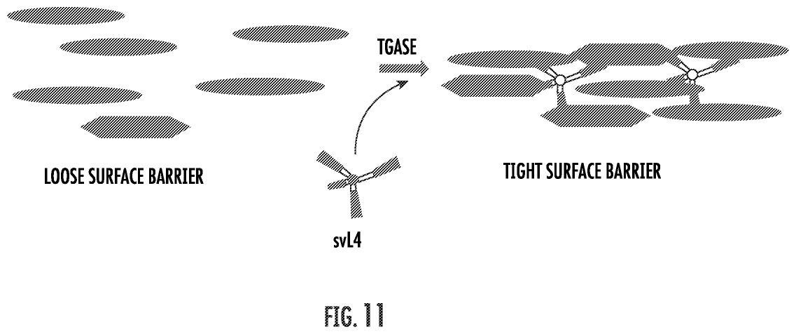

Sequence-specific peptide substrates have been identified for each of the transglutaminase isozymes 1 to 6 (Sugimura et al., 2008; Fukui et al., 2013; Tanabe et al., 2019). These investigators demonstrated the reaction of the enzyme by covalent iso-peptide linkage between a single peptide and a protein (Tanabe et al., 2019). However, a “single,” monovalent peptide will not provide cross-linking of proteins but will only attach a peptide to one protein. The arms of the multivalent structured polypeptides disclosed herein (e.g., svL4) provide attachments to and cross-link multiple proteins (i.e., potentially four proteins for a tetravalent structure). A cross-linked mesh is possible only with the disclosed multivalent peptide which serves as a substrate for TGase. Thus, the present invention provides a multivalent peptide that serves as a substrate for TGase cross-linking activity to restore a tight, functional epidermal surface barrier. This important characteristic of the technology is illustrated in FIG. 11.

The ability of svL4 to restore normal epidermal morphology in this study of murine AD was provided entirely by the amino acids in the N-terminal half of each arm of the tetravalent peptide, which contains two glutamine residues. The glutamine residues provided a substrate for TGases and we propose that the multi-arm structure of the peptide allowed formation of additional cross-links between proteins of the stratum corneum. We therefore propose that these events describe a mechanism of action of the peptide in restoring the tight barrier function of the skin.

We explored whether a ligand of CD301b would provide an effective treatment for AD. CD301a⁺/CD301b⁺ macrophages are the predominant immune cell type in the dermis of mouse skin, comprising 50% of all nucleated cells, with approximately 7% as CD301b⁺ DCs (Dupasquier et al., 2004). Whereas DCs are the primary CD301b (MGL2)-expressing cells, Kanemaru et al. (2019) showed that dermal DCs also express CD301a (MGL1). Similarly, although CD301a is predominantly expressed by murine macrophages, dermal macrophages that are essential for resolution of AD also express CD301b. Kanemaru et al. (2019) concluded that the primary cause of AD in the mouse model was the infiltration of neutrophils, which may also apply to other inflammatory diseases. As depicted in FIGS. 2A-2F, topical application of the peptide svL4 overrides the response of the skin to the irritants SDS and LPS and allows the return to normal morphology, even in the continuous presence of LPS. The primary response to treatment is the reduction in the number of neutrophils in the dermis. Although CD301b⁺ cells did not seem to be a significant factor in the initiation of restoration, macrophages possibly were responsible for phagocytosis of apoptotic neutrophils (Greenlee-Wacker, 2016) during the resolution phase.

### Restoration of the Epidermal Barrier Facilitates Formation of a Ca²⁺ Gradient and the Return to a Normal Epidermal Morphology

Ca²⁺ plays a major role in regulation of homeostasis of the epidermis. A characteristic Ca²⁺ gradient has a peak concentration within the stratum granulosum, with declining concentrations toward the outer stratum corneum and the deeper basal layer (Elias et al., 2002; Mauro et al., 1998). The gradient is composed of extracellular, cytosolic and organelle free Ca′, but only 2% of the stratum granulosum is extracellular space (Celli et al., 2010; Behne et al., 2011). Because the cytosolic Ca²⁺ is usually maintained very low (˜0.1 μM), the average concentration of 10 to 20 μM suggests vast stores of Ca²⁺ in cytoplasmic organelles, i.e., endoplasmic reticulum and Golgi structures. The N-terminal region of profilaggrin contains a S100 domain that binds Ca²⁺ (Osawa et al., 2011), which releases the cation as the protein is degraded.

Whereas media with a low Ca²⁺ concentration (30 μM) promotes proliferation of keratinocytes, higher concentrations (>100 μM) are required for differentiation and formation of the outer cell layers during 3-D reconstruction of the epidermis (Bikle et al., 2012; Lee and Lee, 2018; Lee, 2020; Teshima et al., 2020). Disruption of the barrier function of the stratum corneum dissipates the gradient. Thus, expansion of the epidermis upon induction of dermatitis may result from lower Ca²⁺ in the outer layers and a normal, thin epidermis upon treatment likely reflects keratinocyte differentiation upon restoration of the Ca²⁺ gradient. The acidic stratum corneum may serve to attract the peptide to the epidermal surface (Behne et al., 2002; Hanson et al., 2002). The observation that topical but not subcutaneous application of svL4 supports a role for the physical presence of the peptide at the outer surface of the skin. Restoration of the barrier function and consequently the Ca²⁺ gradient is a prerequisite to terminal differentiation of keratinocytes and the return to normal morphology of the epidermis (Behne et al., 2011; Elsholz et al., 2014; Lee, 2020).

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

All headings are for the convenience of the reader and should not be used to limit the meaning of the text that follows the heading, unless so specified.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Behne M J, Meyer J W, Hanson K M, Barry N P, Murata S, Crumrine D, et al. NHE1 regulates the stratum corneum permeability barrier homeostasis. Microenvironment acidification assessed with fluorescence lifetime imagine. J Biol Chem 2002; 277:47399-406.
Behne M J, Sanchez S, Barry N P, Kirschner N, Meyer W, Mauro T M, et al. Major translocation of calcium upon epidermal barrier insult: imaging and quantification via FLIM/Fourier vector analysis. Arch Dermatol Res 2011; 303:103-15.
Bikle D D, Xie Z, Tu C-L. Calcium regulation of keratinocyte differentiation. Expert Rev Endocrinol Metab 2012; 7:461-72.
Bitton A, Avlas S, Reichman H, Itan M, Karo-Atar D, Azouz N P, et al. A key role for IL-13 signaling via the type 2 IL-4 receptor in experimental atopic dermatitis. Sci Immunol 2020; 5: eaaw2938.
Brown C C, Gudjonson H, Pritykin Y, Deep D, Lavallée V-P, Mendoza A, et al. Transcriptional basis of mouse and human dendritic cell heterogeneity. Cell 2019; 179:846-63.
Celli A, Sanchez S, Behne M, Hazlett T, Gratton E, Mauro. The epidermal Ca²⁺ gradient: measurement using the phasor representation of fluorescent lifetime imaging. Biophys J 2010; 98:911-21.
Chiang C-C, Cheng W-J, Korinek M, Lin C-Y, Hwang T-L. Neutrophils in psoriasis. Front Immunol 2019; 10:2376.
Dupasquier M, Stoitzner P, van Oudenaren A, Romani N, Leenen P J M. Macrophages and dendritic cells constitute a major subpopulation of cells in the mouse dermis. J Invest Dermatol 2004; 123:876-9.
Eckert R L, Sturniolo M T, Broome A-M, Ruse M, Rorke E A. Transglutaminase function in epidermis. J Invest Dermatol 2005; 124:481-92.
Eggink L L, Roby K F, Cote R, Hoober J K. An innovative immunotherapeutic strategy for ovarian cancer: CLEC10A and glycomimetic peptides. J ImmunoTher Cancer 2018; 6:28.
Eggink L L, Spyroulias G A, Jones N G, Hanson C V, Hoober J K. A peptide mimetic of 5-acetylneuraminic acid-galactose binds with high avidity to siglecs and NKG2D. PloS One 2015; 10:e0130532.
Elias P M, Ahn S K, Brown B E, Crumrine D, Feingold K R. Origin of the epidermal calcium gradient: regulation by barrier status and role of active vs passive mechanisms. J Invest Dermatol 2002; 119:1269-74.
Elsholz F, Harteneck C, Muller W, Friedland K. Calcium—a central regulator of keratinocyte differentiation in health and disease. Eur J Dermatol 2014; 24:650-61.
Fukui M, Kuramoto K, Yamasaki R, Shimizu Y, Itoh M, Kawamoto T, Hitomi K. Identification of a highly reactive substrate peptide for transglutaminase 6 and its use in detecting transglutaminase activity in the skin epidermis FEBS J 2013; 280:1420-9.
Fume K, Ito T, Tsuji G, Ulzii D, Vu Y H, Kido-Nakahara M, et al. The IL-13-0VOL1-FLG axis in atopic dermatitis. Immunology 2019; 158:281-6.
Greenlee-Wacker M C. Clearance of apopotic neutrophils and resolution of inflammation. Immunol Rev 2016; 273:357-70.
Griffin M, Casadio R, Bergamini C M. Transglutaminases: natures biological glues. Biochem J 2002; 368:377-96.
Guttman-Yassky E, Nograles K E, Krueger J G. Contrasting pathogenesis of atopic dermatitis and psoriasis—Part 1: Clinical and pathologic concepts. J Allergy Clin Immunol 2011; 127:1110-8.
Hamilton J D, Ungar B, Guttman-Yassky E. Drug evaluation review: dupilumab in atopic dermatitis. Immunotherapy 2015; 7:1043-58.
Hanson K M, Behne M J, Barry N P, Mauro T M, Gratton E, Clegg R M. Two-photon fluorescence lifetime imaging of the skin stratum corneum pH gradient. Biophys J 2002; 83:1682-90.
Harb H, Chatila T A. Mechanisms of dupilumab. Clin Exp Allergy 2020; 50:5-14.
Heger L, Balk S, Lühr J J, Heidkamp G F, Lehmann C H K, Hatscher L, et al. CLEC10A is a specific marker for human CD1c⁺ dendritic cells and enhances their toll-like receptor 7/8-induced cytokine secretion. Front Immunol 2018; 9:744.
Higashi N, Fujioka K, Denda-Nagai K, Hashimoto S, Nagai S, Sato T, et al. The macrophage C-type lectin specific for galactose/N-acetylgalactosamine is an endocytic receptor expressed on monocyte-derived immature dendritic cells. J Biol Chem 2002; 277:20686-93.
Hopp T P, Woods K R. Prediction of protein antigenic determinants from amino acid sequences. Proc Natl Acad Sci USA 1981; 78:3824-8.
Jin H, He R, Oyoshil M, Geha R S. Animal models of atopic dermatitis. J Invest Dermatol 2009; 129:31-40.
Johannessen B R, Skov L K, Kastrup J S, Kristensen O, Bolwig C, Larsen J N, et al. Structure of the house dust mite allergen Der f 2: implications for function and molecular basis of IgE cross-reactivity. FEBS Lett 2005; 579:1208-12.
Kalinin A, Marekov L N, Steinert P M. Assembly of the epidermal cornified cell envelope. J Cell Sci 2001; 114:3069-70.
Kanemaru K, Noguchi E, Tahara-Hanaoka S, Mizuno S, Tateno H, Denda-Nagai K, et al. Clec10a regulates mite-induced dermatitis. Sci Immunol 2019; 4:eaax6908.
Karande P, Jain A, Mitragotri S. Discovery of transdermal penetration enhancers by high-throughput screening. Nat Biotech 2004; 22:192-7.
Kawakami Y, Yumoto K, Kawakami T. An improved mouse model of atopic dermatitis and suppression of skin lesions by an inhibitor of Tec family kinases. Allergol Internatl 2007; 56:403-9.
Kim T-G, Kim S H, Park J, Choi W, Sohn M, Na H Y, et al. Skin-specific CD301b⁺ dermal dendritic cells drive IL-17-mediated psoriasis-like immune response in mice. J Invest Dermatol 2018; 138:844-53.
Kolumam G, Wu X, Lee W P, Hackney J A, Zavala-Solorio J, Gandham V, et al. IL-22R ligands IL-20, IL-22, and IL-24 promote wound healing in diabetic db/db mice. PLOS One 2017; 12:e0170639.
Langan S, Irvine A D, Weidinger. Atopic dermatitis. Lancet 2020; 396:345-60.
Lee S E, Lee S H. Skin barrier and calcium. Ann Dermatol 2018; 30:265-75.
Lee A-Y. Molecular mechanism of epidermal barrier dysfunction as primary abnormalities. Int J Mol Sci 2020; 21:1194.
Liedén A, Winge M C G, Sääf A, Kockum I, Ekelund E, Rodriguez E, et al. Genetic variation in the epidermal transglutaminase genes is not associated with atopic dermatitis. PLOS ONE 2012; 7: e49694.
Liu T, Zhang L, Joo D, Sun S-C. NF-κB signaling in inflammation. Signal Trans Targ Therapy 2017; 2:e17023.
Lowes M A, Suarez-Fariñas M, Krueger J G. Immunology of psoriasis. Annu Rev Immunol 2014; 32:227-55.
Lu Y-C, Yeh W-C, Ohashi P S. LPS/TLR4 signal transduction pathway. Cytokine 2008; 42:145-51.
Martel B C, Lovato P, Baumer W, Olivry T. Translational animal models of atopic dermatitis for preclinical studies. Yale J Biol Med 2017; 90:389-402.
Mauro T, Bench G, Sidderas-Haddad E, Feingold K, Elias P, Cullander C. Acute barrier perturbation abolishes the Ca²⁺ and K⁺ gradients in murine epidermis: quantitative measurement using PIXE. J Invest Dermatol 1998; 111:1198-201.
Menezes M E, Bhoopathi P, Pradhan A K, Emdad L, Das S K, Guo C, et al. Roles of MDA-7/IL-24 a multifunction protein in human diseases. Adv Cancer Res 2018; 138:143-82.
Mitamura Y, Nunomura S, Fume M, Izuhara K. IL-24: a new player in the pathogenesis of pro-inflammatory and allergic skin diseases. Allergol Internatl 2020; 69:405-11.
Myles I A, Fontecilla N M, Valdez P A, Vithayathil P J, Naik S, Belkaid Y, et al. Signaling via the IL-20 receptor inhibits cutaneous production of IL-1β and IL-17A to promote infection with methicillin-resistant Staphylococcus aureus. Nat Immunol 2013; 14:804-13.
Munera-Campos M, Carrascosa J M. Innovation in atopic dermatitis: from pathogenesis to treatment. Actas Dermasifiliogr 2020; 111:205-21.
Nguyen A V, Soulika A M. The dynamics of the skin's immune system. Int J Molec Sci 2019; 20:1811.
Osawa R, Akiyama M, Shimizu H. Filaggrin gene defects and the risk of developing allergic disorders. Allergol Internatl 2011; 60:1-9.
Saini S, Pansare M. New insights and treatments in atopic dermatitis. Ped Clin North Am 2019; 66:1021-33.
Shook B A, Wasko R R, Rivera-Gonzalez G C, Salazar-Gatzimas E, Lopez-Giraldez F, Dash B C, et al. Myofibroblast proliferation and heterogeneity are supported by macrophages during skin repair. Science 2018; 362:909.
Singh S K, Streng-Ouwehand I, Litjens M, Weelij D R, Garcia-Vallejo, van Vliet S J, et al. Characterization of murine MGL1 and MGL2 C-type lectins: distinct glycan specificities and tumor binding properties. Molec Immunol 2009; 46:1240-9.
Smith F J D, Irvine A D, Terron-Kwiatkowski A, Sandilands A, Campbell L E, Zhao Y, et al. Los-of-function mutations in the gene encoding filaggrin cause ichthyosis vulgaris. Nat Genet 2006; 38:337-341.
Su H, Luo Y, Sun J, Liu X, Ling S, Xu B, et al. Transglutaminase 3 promotes skin inflammation in atopic dermatitis by activating monocyte-derived dendritic cells via DC-SIGN. J Invest Dermatol 2020; 140:170-9.
Sugimura Y, Hosono M, Kitamura M, Tsuda T, Yamanishi K, Maki M, Hitomi K. Identification of preferred substrate sequences for transglutaminase 1—development of a novel peptide that an efficiently detect cross-linking enzyme activity in the skin. FEBS J 2008; 275:5667-77.
Tanabe Y, Yamane M, Kato M, Teshima H, Kuribayashi M, Tatsukawa H, et al. Studies on differentiation-dependent expression and activity of distinct transglutaminases by specific substrate peptides using three-dimensional reconstructed epidermis. FEBS J 2019; 286:2536-48.
Teshima H, Kato M, Tatsukawa H, Hitomi K. Analysis of the expression of transglutaminases in the reconstructed human epidermis using a three-dimensional cell culture. Anal Biochem 2020; 603:113606.
Törmä H, Lindberg M, Berne B. Skin barrier disruption by sodium lauryl sulfate exposure alters the expression of involucrin, transglutaminase 1, profilaggrin, and kallikreins during the repair phase in human skin in vivo. J Invest Dermatol 2008; 128:1212-19.
Urban, K, Chu S, Giesey R L, Mehrmal S, Uppal P, Nedley N, Delost G R. The global, regional, and national burden of atopic dermatitis in 195 countries and territories: an ecological study from the Global Burden of Disease Study 2017. JAAD Internat 2021; 2:12-18.
van Kooyk Y, Ilarregui J M, van Vliet S J. Novel insights into the immunomodulatory role of the dendritic cell and macrophage-expressed C-type lectin MGL. Immunobiology 2015; 220:185-92
van Vliet S J, van Liempt E, Geijtenbeek T B H, van Kooyk Y. Differential regulation of C-type lectin expression on tolerogenic dendritic cell subsets. Immunobiology 2006; 211:577-585.
Villani A-C, Saija R, Reynolds G, Sarkizova S, Shekhar K, Fletcher J, et al. Single-cell RNA-seq reveals new types of human blood dendritic cells, monocytes, and progenitors. Science 2017; 356:eaah4573.
Wei J C J, Edwards G A, Martin D J, Huang H, Crichton M L, Kendall M A F. Allometric scaling of skin thickness, elasticity, viscoelasticity to mass for micro-medical device translation: from mice, rats, rabbits, pigs to humans. Sci Rep 2017; 7:15885.
Yang B, Suwanpradid J, Sanchez-Lagunes R, Choi H W, Hoang P, Wang D, et al. IL-27 facilitates skin wound healing through induction of epidermal proliferation and host defense. J Invest Dermatol 2017; 137:1166-75.

1. A method of treating a patient having a neutrophil-driven inflammatory disease, the method comprising:
administering to the patient a multivalent structured polypeptide comprising at least two copies of a therapeutic peptide;
wherein the sequence of the therapeutic peptide consists of the sequence X₁-X₂-X₃-X₄-X₅-X₆-X₇-X₈-NQHTPR (SEQ ID NO: 10) with each of X₁, X₂, X₃, X₄, X₅, X₆, X₇, and X₈ independently being absent or any amino acid residue,
so long as the therapeutic peptide comprises at least 7 amino acid residues and at least one of X₁, X₂, X₃, X₄, X₅, X₆, X₇, and X₈ is Q.

2. The method of claim 1, wherein the therapeutic peptide acts as a substrate for a transglutaminase to induce cross-linking of the stratum corneum, restore the epidermal barrier, and protect the patient from environmental pathogens and allergens.

3. The method of claim 1, further comprising identifying the patient as having a neutrophil-driven inflammatory disease.

4. The method of claim 1, wherein the neutrophil-driven inflammatory disease is atopic dermatitis (AD), psoriasis, or asthma.

5. The method of claim 4, wherein the neutrophil-driven inflammatory disease is AD.

6. The method of claim 1, wherein at least two of X₁, X₂, X₃, X₄, X₅, X₆, X₇, and X₈ are Q and the at least two amino acid residues of Q are separated by two amino acid residues.

7. The method of claim 1, wherein the therapeutic peptide consists of 7 to 12 amino acids.

8. The method of claim 7, wherein the therapeutic peptide comprises VQATQSNQHTPR (SEQ ID NO:1).

9. The method of claim 1, wherein the multivalent structured polypeptide has a central framework, a linker sequence, and at least two arms, wherein each arm comprises the therapeutic peptide, and each arm is linked to the central framework via the linker sequence.

10. The method of claim 9, wherein the linker sequence is selected from the group consisting of: GGGS (SEQ ID NO:3), GGGSGGGS (SEQ ID NO:4), SSSS (SEQ ID NO:5), and SSSSSSSS (SEQ ID NO:6).

11. The method of claim 1, wherein the multivalent structured polypeptide is tetravalent.

12. The method of claim 11, wherein the multivalent structured polypeptide comprises or consists of svL4 (SEQ ID NO:7).

13. The method of claim 1, wherein the multivalent structured polypeptide comprises at least two therapeutic peptides comprising VQATQSNQHTPR (SEQ ID NO:1) and at least one therapeutic peptide comprising NPSHPLSG (SEQ ID NO:2).

14. The method of claim 1, wherein the therapeutic peptide is administered topically.

15. The method of claim 1, wherein the multivalent structured polypeptide is administered to an area where dermatitis is present.

16-18. (canceled)

19. A kit, comprising:
a multivalent structured polypeptide comprising at least two copies of a therapeutic peptide;
wherein the sequence of the therapeutic peptide consists of the sequence X₁-X₂-X₃-X₄-X₅-X₆-X₇-X₈-NQHTPR (SEQ ID NO: 10) with each of X₁, X₂, X₃, X₄, X₅, X₆, X₇, and X₈ independently being absent or any amino acid residue,
so long as the therapeutic peptide comprises at least 7 amino acid residues and at least one of X₁, X₂, X₃, X₄, X₅, X₆, X₇, and X₈ is Q; and
instructions teaching administration of the multivalent structured polypeptide to a patient having a neutrophil-driven inflammatory disease.

20-27. (canceled)

28. A pharmaceutical composition comprising:
a multivalent structured polypeptide comprising at least two copies of a therapeutic peptide;
wherein the sequence of the therapeutic peptide consists of the sequence X₁-X₂-X₃-X₄-X₅-X₆-X₇-X₈-NQHTPR (SEQ ID NO: 10) with each of X₁, X₂, X₃, X₄, X₅, X₆, X₇, and X₈ independently being absent or any amino acid residue,
so long as the therapeutic peptide comprises at least 7 amino acid residues and at least one of X₁, X₂, X₃, X₄, X₅, X₆, X₇, and X₈ is Q; and
a second pharmaceutical intervention, wherein the second pharmaceutical intervention is a corticosteroid and/or a monoclonal antibody.

29. The pharmaceutical composition of claim 28, wherein the multivalent structured polypeptide comprises or consists of svL4 (SEQ ID NO:7).

30. The pharmaceutical composition of claim 28, wherein the second pharmaceutical intervention comprises a topical corticosteroid selected from triamcinolone acetonide, hydrocortisone, and a combination thereof.

31. The pharmaceutical composition of claim 28, wherein the second pharmaceutical intervention comprises an injectable monoclonal antibody selected from the group consisting of dupilumab, nemolizumab, secukinumab, and combinations thereof.


SEQUENCE LISTING (10 sequences)

SEQ ID NO: 1 - protein, 12 aa, Artificial Sequence
  synthetic sequence
VQATQSNQHTPR

SEQ ID NO: 2 - protein, 8 aa, Artificial Sequence
  synthetic sequence
NPSHPLSG

SEQ ID NO: 3 - protein, 4 aa, Artificial Sequence
  synthetic sequence
GGGS

SEQ ID NO: 4 - protein, 8 aa, Artificial Sequence
  synthetic sequence
GGGSGGGS

SEQ ID NO: 5 - protein, 4 aa, Artificial Sequence
  synthetic sequence
SSSS

SEQ ID NO: 6 - protein, 8 aa, Artificial Sequence
  synthetic sequence
SSSSSSSS

SEQ ID NO: 7 - protein, 3 aa, Artificial Sequence
  synthetic sequence
  MISC_FEATURE (1)..(1): VQATQSNQHTPRGGGS linked to the N terminus
  MISC_FEATURE (1)..(1): VQATQSNQHTPRGGGS linked to the end of the side chain lysyl
  MOD_RES (2)..(2): AMIDATION
  MISC_FEATURE (3)..(3): VQATQSNQHTPRGGGS linked to the N-terminus
  MISC_FEATURE (3)..(3): VQATQSNQHTPRGGGS linked to the end of the side chain lysyl
KKK

SEQ ID NO: 8 - protein, 3 aa, Artificial Sequence
  synthetic sequence
  MISC_FEATURE (1)..(1): NQHTPRGGGS linked to the N-terminus
  MISC_FEATURE (1)..(1): NQHTPRGGGS linked to the end of the side chain lysyl
  MOD_RES (2)..(2): AMIDATION
  MISC_FEATURE (3)..(3): NQHTPRGGGS linked to the N-terminus
  MISC_FEATURE (3)..(3): NQHTPRGGGS linked to the end of the side chain lysyl
KKK

SEQ ID NO: 9 - protein, 3 aa, Artificial Sequence
  synthetic sequence
  MISC_FEATURE (1)..(1): NPSHPLSGGGGS linked to the N-terminus
  MISC_FEATURE (1)..(1): NPSHPLSGGGGS linked to the end of the side chain lysyl
  MOD_RES (2)..(2): AMIDATION
  MISC_FEATURE (3)..(3): NPSHPLSGGGGS linked to the N-terminus
  MISC_FEATURE (3)..(3): NPSHPLSGGGGS linked to the end of the side chain lysyl
KKK

SEQ ID NO: 10 - protein, 14 aa, Artificial Sequence
  synthetic sequence
  X (1)..(8): X is equal to any amino acid residue or is absent so long as the sequence comprises at least 7 amino acid residues and at least one amino acid residue preceding asparagine is a glutamine.
XXXXXXXXNQHTPR